(19) United States
(12) Patent Application Publication
(10) Pub. No.: US 2005/0100510 A1
(43) Pub. Date: May 12, 2005

(54) Methods for using gadolinium as a contrast media

(76) Inventor: Frank Falco, Kennett Square, PA (US)

Correspondence Address: FOX ROTHSCHILD O'BRIEN & FRANKEL LLP;PRINCETON PIKE CORPORATE CENTER, 997 LENOX DRIVE, BUILDING 3, LAWRENCEVILLE, NJ, 08648, US

(21) Appl. No.: 10/840,828

(22) Filed: May 8, 2004

Related U.S. Application Data
(60) Provisional application No. 60/469,459, filed on May 9, 2003.
(60) Provisional application No. 60/477,411, filed on Jun. 9, 2003.

(51) Int. Cl. A61K 49/00

(52) U.S. Cl. 424/9.36

(57) ABSTRACT

Disclosed herein are methods of using gadolinium as a contrast substance. Preferred embodiments of the method use gadolinium to localize placement of a needle for an injection of a substance. The preferred method includes injecting gadolinium into a target portion of a spinal area to create a contrast between the target portion and surrounding tissue and fluoroscopically creating a visualization of the contrast for guiding injection into the target portion of at least one of a diagnostic substance and a therapeutic substance. At least one of the diagnostic substance and the therapeutic substance is preferably then injecting into the target portion. In some aspects of the preferred embodiment, the placement of the needle is adjusted prior to injecting into the target portion at least one of the diagnostic substance and the therapeutic substance. Additional methods are disclosed herein, including for example, methods of using gadolinium to facilitate lumbar discography

### CROSS-REFERENCE TO RELATED APPLICATIONS

The present application claims the benefit under 35 U.S.C. § 119(e) of U.S. Application No. 60/469,459, filed May 9, 2003, and 60/477,411, filed Jun. 9, 2003, both of which are hereby incorporated by reference in their entirety for all purposes.

### BACKGROUND OF THE INVENTION

1. Field of Invention

The invention disclosed herein relates generally to methods for using gadolinium as a contrast media in the spinal area. More specifically, preferred embodiments of the disclosed invention relate to methods of using gadolinium to facilitate the localization of injection of a substance into a target portion of a spinal area, methods of using gadolinium to visualize spinal anatomy and methods of using gadolinium to facilitate lumbar discography.

2. Description of the Related Art

Contrast media, also referred to as contrast agents and/or contrast substances, have traditionally been used to assist medical professionals in obtaining visualizations of internal portions of the human body. Some of the more ferrous contrast substances are receptive to magnetic resonance imaging (MRI) due to the their ability to respond to magnetism, while other contrast substances, due to their ability to absorb radiation, are receptive to x-ray technologies such as computed axial topography (CAT) and other fluoroscopic devices. The suitability of a method of imaging (e.g. x-ray based imaging, magnetic-based imaging, etc.) is at least in part dependent upon the type of tissue being imaged. Consequently, the suitability of a particular contrast substance is a function at least in part of the ability of the contrast substance to respond to the type of imaging that is appropriate for the type of tissue being imaged. These varying levels of radiation absorption and/or magnetic response are what facilitate imaging of the inside of the human body.

Iodine is the most common contrast substance used for the soft tissue fluoroscopic imaging of spinal areas, due to its heightened ability to absorb radiation. There is a substantial concern, however, about the potential complications associated with iodine-based contrast substances, such as allergic reactions to the iodine contrast. The most serious of potential contrast substance allergies is anaphylaxis, which occurs in between about one and two percent of first dose administrations of iodine contrast substance, and increases to about seventeen to thirty-five percent with repeated iodine exposure in individuals who demonstrate a sensitivity to iodine contrast substance. These allergies can preclude the patient from undergoing spinal injection procedures for the diagnosis and treatment of painful spinal disorders. In particular, lumbar discography is a procedure that requires administration of relatively large volumes of a contrast substance into multiple spinal discs. An alternative to iodine contrast substances is clearly needed to enhance the widespread utility of contrast substances for the spinal area.

Conventional discography is a procedure that has used non-iodinic iodinated contrast substances to obtain visualizations of spinal disc internal architecture in the setting of chronic axial low back pain. Although conventional discography using newer contrast substances such as iohexol or iopamidol is relatively safer, the risk of anaphylactic reactions with administration of iohexol and/or iopamidol contrast substances is still between about one percent and two percent, increasing to between about seventeen percent and thirty-five percent with repeated exposure in patients sensitive to these contrast substances. Prophylactic measures against anaphylactic reactions during interventional pain management procedures have been described and used in clinical practice. Nevertheless, however, there is a subset of patients with chronic axial low back pain who are not offered discography, or who elect not to undergo the procedure because of iodinated contrast material allergy.

Gadolinium is a ferrous material that responds well to magnetic imaging. The use of intradiscal gadolinium during discography, in conjunction with post-discography magnetic resonance imaging is one of the current methods of imaging used in discography. This method is a successful method for visualization of spinal disc architecture in the setting of iodinated contrast allergy or in patients who wish to limit radiation exposure, however spinal imaging using magnetic resonance imaging can be inadequate because magnetic resonance imaging has a tendency to magnify bulging tissues, thereby causing in inappropriate interpretation of spinal disc problems. Another limitation of magnetic resonance imaging is the fact that it is obtained with the patient lying down in complete immobility. In degenerative disc conditions that create stenosis and instability, magnetic resonance imaging merely offers a static view. However, very often stenosis, nerve root compression and symptoms of other condition may only be observable during motion (walking, turning in bed, etc.). Therefore other tests may be necessary because the compression or instability may not fully show up on a visualization created by magnetic resonance imaging.

In some cases, magnetic resonance imaging cannot be used due to the effects of the magnetic field on the functionality of pacemakers or other devices. Patient size or claustrophobia may also be a limitation. Furthermore, while use of magnetic resonance imaging is advantageous for most types of tissue, magnetic resonance imaging is suboptimal in degenerated conditions, or areas of infection or previous spinal surgery. Additionally, it should be noted that magnetic resonance imaging may not offer reliable images if certain instrumentation is present in the spinal area. Coupled with the potentially harmful side-effects of iodinated contrast substances, the need is underscored for a contrast substance that can be used in the spinal area that can be identified using fluoroscopic scanning, rather than magnetic-based scanning.

### SUMMARY OF THE INVENTION

Disclosed herein is a method of using gadolinium that preferably includes injecting gadolinium into a target portion of the spinal area to create a contrast between the target portion and surrounding tissue. The method of using gadolinium preferably includes fluoroscopically identifying the contrast to provide guidance for the injection into the target portion of a substance, preferably a diagnostic substance and/or therapeutic substance. The target portion preferably includes any one or more of the a sacroiliac joint, a lumbar, a cervical disc, an intervertebral disc, a facet joint, a nerve root, a sympathetic nerve, an epidural space, an intrathecal space and another spinal area portion.

As used herein, the terms “fluoroscope”, “fluoroscopic”, “fluoroscopy”, “fluoroscopically, etc. are used in their broadest sense to refer to any and all imaging devices that utilize X-rays or other electromagnetic waves to create a visualization of a contrast. For the purpose of a nonlimiting example, this includes computed topography (CT) scans, computed axial topography (CAT) scans, X-ray machines, AP imaging, etc.

Also disclosed herein is a method of using gadolinium to facilitate the localization of injection of a substance into a target portion of a spinal area, comprising injecting gadolinium into the target portion to create a contrast between the target portion and surrounding tissue. In some aspects, this includes fluoroscopically identifying the contrast to provide guidance for the injection of the substance into the target portion, and in some aspects, includes inserting the needle into the target portion.

Another preferred method of the invention uses gadolinium to localize placement of a needle for an injection of a substance. The method preferably includes injecting gadolinium into a target portion of a spinal area to create a contrast between the target portion and surrounding tissue and fluoroscopically creating a visualization of the contrast for guiding injection into the target portion of at least one of a diagnostic substance and a therapeutic substance. A “visualization” comprises any visually perceptible representation, regardless of the medium on which it is displayed. A two-dimensional photograph and a three-dimensional computer display are both nonlimiting examples of a visualization. Any reference herein to a specific type of visualization (e.g. “a CAT scan”) is interchangeable with any other suitable fluoroscopy-based visualization. The visualization is fluoroscopically created by preferably identifying the contrast and then creating the visualization from the contrast.

At least one of the diagnostic substance and the therapeutic substance is preferably injected into the target portion. In some aspects, the placement of the needle is adjusted prior to injecting into the target portion the diagnostic and/or thereapeutic substance. The target portion preferably comprises a sacroiliac joint, a lumbar disc, a cervical disc, an intervertebral disc, a facet joint, a nerve root, a sympathetic nerve, an epidural space, an intra-thecal space and/or another spinal area portion.

Also disclosed herein is a method of using a fluoroscopic device to facilitate localization of injection of a substance into a target portion of a spinal area. The method preferably comprises identifying a gadolinium-induced contrast between the target portion of the spinal area and surrounding tissue, and creating a visualization of the contrast to guide insertion of the needle into the target portion.

Another preferred embodiment of the invention uses gadolinium to visualize spinal anatomy. The method includes injecting gadolinium into at least one spinal disc to create a fluoroscopically perceptible contrast between each of the at least one spinal discs and surrounding tissue. In some aspects a visualization of the contrast is created.

Also disclosed herein is a preferred method of using gadolinium to visualize spinal anatomy. The method preferably includes injecting gadolinium into at least one spinal disc, and using fluoroscopy to create a visualization of the contrast between each of the at least one spinal discs and surrounding tissue. Computer tomography is the preferred method of fluoroscopy. In some aspects, the method preferably includes injecting additional gadolinium into a spinal disc experiencing gadolinium runoff to indicate an enhanced contrast and using fluoroscopy to create an enhanced visualization from the enhanced contrast.

Another preferred embodiment of the invention uses gadolinium to facilitate lumbar discography. The method preferably includes injecting gadolinium into at least one spinal disc to create a contrast between each of the at least one spinal discs and surrounding tissue. Computer tomography is preferably used to identify the contrast and create from the contrast at least one of a two-dimensional visualization and a three-dimensional visualization. In the some embodiments, such as when there is gadolinium runoff or other circumstances, the method further includes injecting additional gadolinium into a spinal disc experiencing gadolinium runoff to enhance the quality of the contrast. Computer tomography is preferably used to identify the enhanced contrast and create from the enhanced contrast at least one of a two-dimensional enhanced visualization and a three-dimensional enhanced visualization.

### BRIEF DESCRIPTION OF THE DRAWINGS

The accompanying drawings, which are incorporated in and form a part of the specification, illustrate the embodiments of the present invention and, together with the description serve to explain the principles of the invention. In the drawings:

FIG. 1 is a flow chart showing an embodiment of a preferred method of using gadolinium; and

FIG. 2 is a flow chart showing an embodiment of another preferred method of using gadolinium.

### DETAILED DESCRIPTION OF THE INVENTION

In describing a preferred embodiment of the invention illustrated in the drawings, specific terminology will be used for the sake of clarity. However, the invention is not intended to be limited to the specific terms so selected, and it is to be understood that each specific term includes all technical equivalents which operate in a similar manner to accomplish a similar purpose.

Gadolinium is a water soluble, non-iodinated contrast substance that is distributed in extracellular fluid and is best known for heightened ferric properties which enhance magnetic resonance imaging. As compared to the risks discussed-above with regards to iodine, gadolinium has also been used safely as a contrast substance in other imaging applications with there being only a 0.06% adverse reaction rate and a 0.0003% to 0.01% severe life-threatening allergic reaction rate to intravenous administration of gadolinium. There is little difference in image quality when comparing gadolinium contrast substances to iodinated contrast substances. Laboratory phantom experiments have demonstrated that similar volumes of gadolinium and iodine exhibit image contrast virtually equal to each other, indicating that imaging is not compromised when using one contrast substance over the other.

There are four gadolinium chelates currently available for administration worldwide. The four gadolinium chelates currently being used are gadopentate dimeglumine (“Magnevist” from Berlex Laboratories, Wayne, N.J.; and Schering, Berlin Germany), gadodiamide (Omniscan; Nycomed, Princeton, N.J.), gadoteridol (ProHance; Bracco Diagnostic, Princeton, N.J.) and gadoversetamide (OptiMARK; Mallinckrodt, St. Louis, Mo.). These chelates exhibit similar pharmacologic characteristics and cannot be differentiated on the basis of adverse reactions.

Although gadolinium retains some of its important ferrous characteristics in soft tissue, it is the ability of gadolinium to absorb radiation that causes an opacification such that a visual representation is created from a fluoroscopically readable contrast. The present invention overcomes the limitations that exist using the current discography methodology utilizing magnetic resonance imaging by providing a method wherein gadolinium is used as the basis for a contrast substance that is preferably injected through the skin and muscles of the back directly into the soft tissue. In discography, for example, the injection of the gadolinium contrast substance would be made directly into the intervertebral discs. This is preferably followed by a CT scan, which provides a detailed 2-dimensional or 3-dimensional visualization of the spinal anatomy. The ability to visualize the details of the structures in the spine using gadolinium will thus allow for a proper medical diagnosis.

Preferred methods of the present invention are useful for visualization of spinal anatomy in a timelier manner than the currently used procedures with magnetic resonance imaging. In preferred embodiments, a method for lumbar discography is provided wherein gadolinium is injected into the spinal discs and is followed by a CT scan or other fluoroscopic imaging to create a clear visualization for analysis of the spinal discs or for other reasons. The CT scans are performed early or late after completion of the lumbar discography, therein allowing for the proper diagnosis in a patient suffering from a spinal condition and in directing effective medical care to that patient.

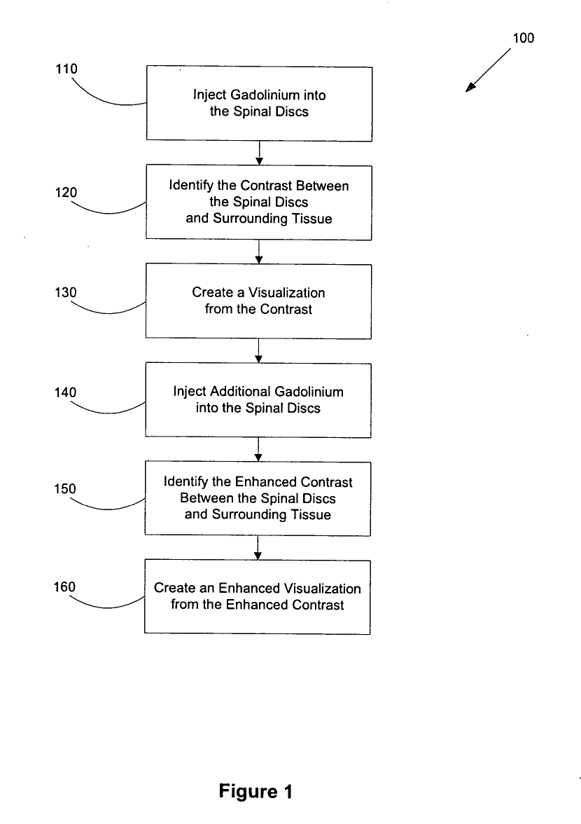

With principal reference to FIG. 1, a preferred embodiment of a method using gadolinium to facilitate lumbar discography is shown and designated generally as 100. Lumbar discography generally refers to an injection technique used to evaluate patients with back pain who, in most cases, have not responded well to extensive conservative care. The most common use of discography is during the preparation and evaluation stage prior to lumbar fusion.

At step 110, gadolinium is injected into the spinal discs to create a contrast between the spinal discs and surrounding tissue. The spinal discs and surrounding tissues are then scanned, preferably utilizing computed tomography. The fluoroscopic device identifies, at step 120, the contrast between the spinal discs and surrounding tissue and, at step 130, creates a visualization of the contrast. This visualization is preferably displayed on a medium of choice, such as paper (e.g. photographic paper) and/or digital rendering (e.g. a computer display screen). The visualization may be two dimensional or three-dimensional.

In some cases, a spinal disc may experience gadolinium runoff and it may be desirable to inject additional gadolinium. At step 140, additional gadolinium is injected into the spinal disc experiencing gadolinium runoff to enhance the quality of the contrast and the spinal discs and surrounding tissues are scanned, preferably using computed tomography. At step 150, the enhanced contrast is identified and an enhanced visualization is then created at step 160 from the enhanced contrast. Similar to a visualization, an enhanced visualization is preferably displayed on a chosen medium, such as paper (e.g. photographic paper) and/or digital rendering (e.g. a computer display screen). The visualization may be two dimensional or three-dimensional.

A first set of test cases was conducted wherein it was identified that gadolinium would adequately opacify, thereby allowing adequate fluoroscopic imaging for of soft tissue portions of the spinal area and, for example, for lumbar discography procedures. The test cases also showed that gadolinium can be used as a viable alternative to nonionic iodinated contrast substance. During the first set of test cases, a solution of 469.01 mg/ml gadolinium (Magnevist—Berlex Laboratories, Wayne, N.J.) with 25 mg/ml Cefazolin (Apothecon, Princeton, N.J.) for antibacterial prophylaxis was injected into each disc using a standard three milliliter syringe. The following is a brief synopsis of six tests from the first set of tests:

Case 1-1

A forty-three-year-old female patient was scheduled for lumbar discography after presenting chronic axial low back pain, as a result of a slip and fall, that had failed to improve with time. Conservative measures such as chiropractic treatments and physical therapy had been unsuccessful. A history before the procedure revealed an anaphylactic reaction to shellfish/iodine that precluded use of iodinated contrast substances during the procedure. Although medical prophylaxis against anaphylaxis was an option in this case, it was decided to proceed with discography using gadolinium as a contrast substance.

The volume of contrast injected at pain onset, the end point (nucleography), the pain intensity measured with the numerical analog scale, and the concordant and/or discordant pain response were recorded for each spinal disc to be injected with gadolinium. Each spinal disc then was visualized with c-arm fluoroscopy to create a visualization of the nucleogram and provocation of the spinal discs. There were no complications during or after the procedure, and adequate pain provocation occurred during the study.

The lateral fluoroscopic image taken after injection of gadolinium into the spinal discs showed gadolinium to be fluoroscopically visible in the disc material, particularly in the spinal discs without annular disruption. The spinal discs with annular disruption tended to clear some of the gadolinium shortly after the injection, this being referred to herein as “gadolinium runoff.” However, the contrast was still of a quality high enough so that a visualization could be created showing the internal disc disruption. When a spinal disc experienced gadolinium runoff, the quality of the contrast was enhanced by injecting additional gadolinium, which in turn facilitated the creation of an enhanced visualization.

A visualization of the lumbar discs was created with the aid of computer axial tomography approximately one-hundred-and-sixty minutes after injection and the contrast created by the gadolinium injection into the L3-L4 spinal disc, was visible on a visualization created by computer axial tomography. At the L4-L5 spinal disc, a disrupted spinal disc, the visualization of the contrast was less intensive, but still visible. Annular disruption at this level was not clearly defined, but the gadolinium-induced contrast did track toward the right posterolateral annulus. At the LS-S1 spinal disc, the lumbosacral angle did not allow for horizontal cuts through the disc material, so the contrast pattern was not completely visualized in this disc.

Case 1-2

A forty-six-year-old female patient was scheduled for lumbar discography because of chronic axial low back pain. The patient of case one-two was allergic to iodinated contrast material. Once again, in lieu of prophylactic measures for contrast allergy, gadolinium was chosen to facilitate visualization of the structure of the spinal discs and provocation of the spinal disc for pain. There was adequate pain provocation and no complications occurred. Discography was performed in a manner similar to case one-one.

Fluoroscopic imaging at the time of disc injection was suboptimal in both the anteroposterior and lateral views because of body habitus. Visualization of the contrast at the L3-L4 spinal disc showed a very distinct normal nucleogram without annular disruption. Due to annular disruption, the L4-L5 and L5-S1 spinal discs retained only very small amounts of gadolinium, experiencing gadolinium run-off. However, injection of additional gadolinium enhanced the quality of the contrast facilitating the creation of an enhanced visualization. The patient of case one-two was taken directly to the computer axial tomography scanner after the procedure for more definitive visualization of gadolinium spread through the disc material. Computer axial tomography images of the lumbosacral spine taken thirty minutes after completion of the procedure showed gadolinium in each disc with an intensity similar to that for the patient of case one-one.

Case 1-3

A thirty-five-year-old female patient was scheduled for lumbar discography because of chronic axial low back pain that had failed to improve with conservative care. A history before the procedure showed anaphylaxis with administration of iodinated contrast material. Because of the severe nature of her allergy, and because the patient had not had prophylaxis before the procedure, it was decided to proceed with gadolinium-based discography followed by a post-procedure scan.

Discography was performed in the same manner as described for the patient of case one-one and there were no complications. Adequate pain provocation was accomplished during the study. The discs were injected with gadolinium and fluoroscopic imaging at the time of injecting the gadolinium showed a distinct normal nucleogram of the L3-L4 spinal disc without annular disruption. Similar to the patients of cases one-one and one-two, annular disruption resulted in gadolinium run-off and a faint nucleographic pattern with posterior and anterior epidural contrast at the L4-L5 spinal disc. However, injection of additional gadolinium enhanced the quality of the contrast facilitating the creation of an enhanced visualization. The L5-S1 spinal disc showed a darker nucleogram with extension of contrast into a posterior annular tear. Visualization and/or enhanced visualizations of the nucleographic patterns in the contrast showed that L3-L4 spinal disc had a normal nucleogram, whereas the L4-L5 spinal disc had minimal nuclear and posterior annular gadolinium. The L5-S1 spinal disc showed a normal nucleogram with posterior annular contrast.

Case 1-4

A thirty-eight-year-old male patient was scheduled for discography because of chronic axial low back pain that failed to respond to conservative care. The patient of case one-four had a history of respiratory distress with administration or consumption of iodinated compounds. Because the case one-four patient had not received prophylaxis against iodine contrast allergy before the procedure, it was decided to proceed with gadolinium discography followed by a post-procedure CT scan.

Gadolinium discography was performed in a manner virtually identical to that for the patient of case one-one. No complications occurred during the study, and there was adequate pain provocation. Fluoroscopic images obtained during the procedure showed distinct, dark, normal nucleograms at the L3-L4, L4-L5 and LS-S1 spinal discs. None of the three spinal discs had annular disruption. This procedure was performed in a hospital short-procedure unit and the patient had a CT scan approximately ninety minutes after the procedure was completed. Visualizations of the L4-L5 and LS-S1 spinal discs showed distinct, normal nucleograms without annular contrast.

The visualization showed three spinal discs without annular disruption and normal nucleograms. Optimal nuclear signal intensity on fluoroscopy (e.g. CT) visualizations, as well as minimal gadolinium runoff, was observed in this setting, as compared with spinal discs that have annular disruption, for example. The presence of an annular disruption may cause gadolinium runoff, such that the nuclear signal observed in the visualization of an annular disruption may be less intense than that found in a normal spine. Thus, if a more intense signal is desired, injection of additional gadolinium would allow for a more accurate, enhanced nucleogram of spinal discs with annular disruption.

The test cases in the first set show that gadolinium-induced contrasts will detail bone structures in the spine, as well as reproduce/provoke the patient's disc pain. Furthermore, post-discography visualizations (e.g. nucleograms, etc.) using computed tomography scans are quicker, cheaper, and are more readily available imaging modality than magnetic resonance imaging. Gadolinium does not require on-site magnetic imaging capabilities. In this respect, medical practices can take advantage of gadolinium-induced contrasts and visualization even if they do not posses magnetic imaging technology. Each test case in the first series showed that gadolinium is clearly visible on fluoroscopy-based imaging devices. In particular, spinal discs with and without annular disruption are still visible on a CT scan more than one-hundred-and-twenty minutes after injection of the gadolinium.

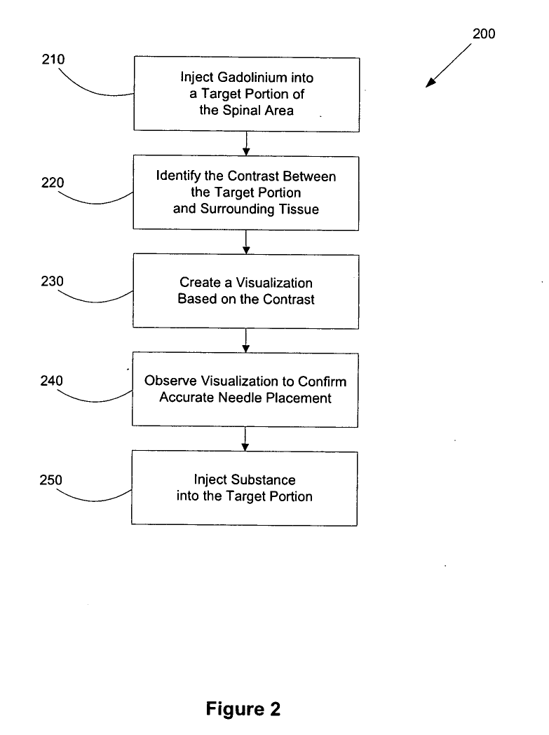

With principal reference to FIG. 2, another preferred embodiment of a method of using gadolinium is shown and designated generally as 200. The method relates to the use of gadolinium to localize placement of a needle for an injection of a substance. At step 210, gadolinium is injected into a target portion of a spinal area to create a contrast between the target portion and surrounding tissue. This target portion is usually a soft tissue portion of the spinal area. For the purpose of illustration without limitation, this target portion may include the sacroiliac joint, lumbar, cervical discs, an intervertebral disc, facet joints, nerve roots, sympathetic nerves, epidurals, intra-thecal spaces and other soft tissue in the spinal area.

At step 220 and step 230, imaging technology, such as a computerized topography scan or other fluoroscopic device, creates a visualization of the contrast between the target portion and surrounding tissue for guiding insertion of the needle into the target portion. In preferred embodiments, scanning the visualization comprises identifying the contrast in the spinal area at step 220 and creating the visualization at step 230. This is preferably accomplished with a CAT scan, which scans the spinal area and creates a three-dimensional rendering of the target portion and the surrounding issue, having a contrast between the target portion the surrounding tissue. At step 240, the visualization is then reviewed to confirm that the needle is accurately placed. If the needle is accurately placed, then the substance is then injected into the target portion at step 250, otherwise the placement of the needle is adjusted.

In preferred embodiments, the same needle that injected the gadolinium stays within the target portion that had been injected. Upon confirmation, that the needle is in fact within the target portion, then the substance is injected through the same needle. In some embodiments multiple needles may be used, said embodiments being contemplated by the current invention.

In conducting a second set of test cases, it was confirmed that gadolinium can be used to provide adequate imaging for spinal injection procedures under fluoroscopy without experiencing a lack of suitable opacification. The methods disclosed herein present a viable alternative to nonionic iodinated contrast substance, where gadolinium is preferably used as the contrast substance, thus presenting a long sought, yet unfulfilled need for a safe and viable alternative to nonionic iodinated contrast materials for performing spinal injection procedures.

During the second of set of tests, gadolinium was injected into a variety of target portions of the spinal area to create a contrast between the target portion and surrounding tissue. As described below, these contrasts were adequately sensed using fluoroscopy to create visual representations of the contrast between the target portion and the surrounding tissue. The second set of tests utilized a preferred embodiment of the method of using gadolinium as a contrast substance, where a visualization was created to use as a guide for the accurate injection of a diagnostic or therapeutic substance into the target portion of the spinal area. In this respect, visualization of the contrast helps to localize placement of a needle for an injection of a substance avoiding the harm that the injection might cause to surrounding tissue.

The second set of tests comprised chart review, performance of spinal injections with gadolinium under fluoroscopic guidance, and post procedure follow-up by phone at two and twelve weeks to assess any adverse reactions to the gadolinium. There were no complications during or immediately after the procedures and there were no side effects or complications from the injections. The tests utilized a contrast substance that was 469.01 mg/ml gadolinium (Magnevist—from Berlex Laboratories, Wayne, N.J.). Magnevist was used during all of the procedures in the second set of tests to create fluoroscopic visualizations for assisting in the proper needle placement for a subsequent injection of a diagnostic and/or therapeutic substance. The following is a brief synopsis of six tests from the second set of tests:

Case 2-1

A fifty-two-year-old female patient was scheduled for cervical facet joint injection for chronic neck pain. The patient of case one-two had a history of anterior cervical decompression and fusion at the C5/6 and C6/7 levels. Physical exam findings were suggestive for facet joint dysfunction and pain at the bilateral C5/6 levels. The patient of case two-one had failed conservative treatments with physical therapy, modalities and medications. Due to a history of rheumatic heart disease, the patient of case two-one was premedicated with intravenous Cefazolin (Apothecon, Princeton, N.J.). The patient of case two-one then underwent bilateral C5/6 facet joint injections with 0.25 cubic centimeters gadolinium for joint localization. Visualization of the contrast between the facet joint and the surrounding tissue was then obtained using fluoroscopic imaging, and with the aid of the visualization created therefrom, a needle was inserted into each facet joint and a 0.75 cubic centimeters volume of a mixture of one percent preservative free Lidocaine with forty milligram per cubic centimeter triamcinilone (50:50 ratio) was injected into the facet joints.

Case 2-2

A sixty-three-year-old male patient was diagnosed with a left S1 radiculopathy and complained of persistent left sided lower back pain, despite medications and physical therapy. Needle placement within the left S1 neural foramen and left L5/S1 facet joint was confirmed as accurate using a fluoroscopically created visualization of the contrast between the foramen, L5/S1 facet joint and surrounding tissue. The patient of case two-two then underwent localized injections in both the left S1 nerve root and left L5/S1 facet joint with a solution containing 0.5% preservative free Marcaine with forty milligrams per cubic centimeter triamcinilone (50:50 ratio).

Case 2-3

A forty-three-year-old female patient was assessed for treatment of chronic back pain and segmental leg symptoms localizing in a left L5 distribution. Gadolinium was injected into the neural foramen of the patient of case two-three to create a contrast between the nerve root and the surrounding tissue and a visualization was created using fluoroscopic imaging. A L5 selective nerve root block was then performed on the patient of case two-three using the visualization as a guide to ensure that subsequent injections was safely made. A two cubic centimeter solution containing 0.5% preservative free Marcaine with forty milligrams per cubic centimeter triamcinilone (50:50 ratio) into the L5/S1 foramen.

Case 2-4

A thirty-five-year-old male patient with chronic neck pain and numbness in a right C6 dermatome, underwent a right C6 selective nerve root block using a visualization of a gadolinium-induced contrast of the right C6 nerve that acted a guide to confirm that the needle was accurately inserted. A two cubic centimeter solution of one percent preservative free Lidocaine and triamcinilone was injected into the C5/6 foramen.

Case 2-5

A forty-seven-year-old male patient with low back pain and associated paresthesias in the right lower extremity was evaluated for an interlaminar lumbar epidural steroid injection. The location of the epidural space was confirmed by injecting two cubic centimeters of gadolinium and using fluoroscopic technology to create an epidurogram visualization of the contrast between the epidural space and surrounding tissue. Observing the location of the epidural space on the visualization, a right paramedian approach was used to perform the epidural. A six cubic centimeter solution was used, containing four cubic centimeters of one percent preservative free Lidocaine with two cubic centimeters of forty milligram per cubic centimeter triamcinilone.

Case 2-6

A thirty-three-year-old female history with a nine-month history of persistent axial low back pain had failed all means of conservative treatment. Although a lumbar spine MRI revealed disc desiccation at the L4/5 level, lumbar discography conducted with the guidance of a fluoroscopically-created visualization of a gadolinium-induced contrast uncovered a L4/5 posterior annular tear with reproduction of her concordant low back pain.

The second set of tests showed that gadolinium was visible under fluoroscopy, thereby demonstrating viability as a fluoroscopic contrast substance in the soft issue of the spinal area expanding beyond application to lumbar discography procedure. None of the individuals in the case series of the present invention had any adverse reactions to the gadolinium used to perform the injections. Gadolinium can thus be used to, among other things, facilitate the safe performance of various spinal injection procedures and it presents a good alternative to nonionic iodinated contrast materials.

The results of the tests also indicate that gadolinium is associated with a lower adverse reaction rate and reduced risk of anaphylaxis compared to iodinated contrast media. Injections were performed in target portions of the spinal area in individuals without any known history of iodine contrast allergy. The use of gadolinium in performing spinal injection procedures was found to be both effective and safe in both sets of test cases. In those individuals who have a history of iodine allergy or anaphylaxis, gadolinium is an alternative medium that allows for successful contrast enhancement in performing fluoroscopically guided spine injection procedures without the significant risk of complications associated with iodine-based contrasts.

In a third set of cases, two-hundred-and-thirty patients underwent spinal interventional procedures with fluoroscopic guidance. Two-hundred-and-eleven patients were subsequently evaluated for any adverse reaction in the period immediately after the procedure and up to several weeks after the procedure. Seventy-four of the patients were male and one-hundred-and-thirty-seven were female. The age range of the patient population varied from sixteen years old to eighty-one year old, and the average age of the patient population was 48.7 years old. The only prophylactic medications that were administered before the procedure were antibiotics for patients undergoing discography, patients with significant cardiac disease, or patients with surgical implants. The number and type of procedures along with the number of adverse reactions for each procedure type are summarized in below in Table 1:

TABLE 1

| | Number of | Number of |
|---|---|---|
| Type of Procedure | Procedures | Adverse Reactions |
| Sacroiliac joint | 59 | 2 |
| Lumbar SNRB | 55 | 4 |
| Lumbar interlaminar | 37 | 1 |
| Caudal epidural | 16 | 0 |
| Cervical SNRB | 14 | 1 |
| Lumbar discography | 11 | 0 |
| Cervical interlaminar | 6 | 1 |
| Lumbar sympathetic | 4 | 0 |
| Lumbar epidurolysis | 4 | 0 |
| Lumbar intraarticular facet | 3 | 0 |
| Other miscellaneous | 13 | 1 |

After further study, it was determined that only five (out of the ten) adverse reactions were attributable to allergic-type reactions. Only one of these five adverse reasons were serious, however the serious adverse reaction was determined to be likely due to contributing factors. All of the procedures produced adequate visualizations from gadolinium-induced contrasts created by fluoroscopic devices. The visualizations were of a higher quality with oblique and lateral views during interlaminar epidural injections compared to AP views. The third series of tests further confirmed that gadolinium is a safe and effective alternative to iodine-based contrasts for fluoroscopic imaging of the spinal region.

Although there has been hereinabove described methods for using gadolinium as a contrast media in the spinal area, in accordance with the present invention and for the purposes of illustrating the manner in which the invention may be used to advantage, it should be appreciated that the invention is not limited thereto. Accordingly, any and all modifications, variations, or equivalent arrangements which may occur to one skilled in the art should be considered to be within the scope of the present invention as defined in the appended claims.

1. A method of using gadolinium, comprising injecting gadolinium into a target portion of a spinal area to create a fluoroscopic contrast between the target portion and surrounding tissue.

2. The method of claim 1, comprising fluoroscopically identifying the contrast to provide guidance for injection of a substance into the target portion.

3. The method of claim 2, comprising injecting into the target portion at least one of a diagnostic substance and a therapeutic substance.

4. The method of claim 1, wherein injecting gadolinium into the target portion comprises injecting gadolinium into at least one of a sacroiliac joint, a lumbar disc, a cervical disc, an intervertebral disc, a facet joint, a nerve root, a sympathetic nerve, an epidural space, an intra-thecal space and another spinal area portion.

5. A method of using gadolinium to facilitate the localization of injection of a substance into a target portion of a spinal area, comprising injecting gadolinium into the target portion to create a contrast between the target portion and surrounding tissue.

6. The method of claim 5, comprising fluoroscopically identifying the contrast to provide guidance for the injection of the substance into the target portion.

7. The method of claim 6, comprising injecting into the target portion at least one of a diagnostic substance and a therapeutic substance.

8. A method of using gadolinium to localize placement of a needle for an injection of a substance, comprising:
injecting gadolinium into a target portion of a spinal area to create a contrast between the target portion and surrounding tissue;
fluoroscopically creating a visualization of the contrast for guiding injection into the target portion of at least one of a diagnostic substance and a therapeutic substance;
injecting into the target portion at least one of the diagnostic substance and the therapeutic substance.

9. The method of claim 8, comprising adjusting the placement of the needle prior to injecting into the target portion at least one of the diagnostic substance and the therapeutic substance.

10. The method of claim 8, wherein fluoroscopically creating comprises:
identifying the contrast; and
creating the visualization from the contrast.

11. The method of claim 8, wherein injecting gadolinium into the target portion comprises injecting gadolinium into at least one of a sacroiliac joint, a lumbar disc, a cervical disc, an intervertebral disc, a facet joint, a nerve root, a sympathetic nerve, an epidural space, an intra-thecal space and another spinal area portion.

12. A method of using a fluoroscopic device to facilitate localization of injection of a substance into a target portion of a spinal area, comprising:
identifying a gadolinium-induced contrast between the target portion of the spinal area and surrounding tissue; and
creating a visualization of the contrast to guide injection of the substance into the target portion.

13. A method of using gadolinium to visualize spinal anatomy, comprising injecting gadolinium into at least one spinal disc to create a fluoroscopically perceptible contrast between each of the at least one spinal discs and surrounding tissue.

14. The method of claim 13, comprising creating a visualization of the contrast.

15. A method of using gadolinium to visualize spinal anatomy, comprising:
injecting gadolinium into at least one spinal disc; and
using fluoroscopy to create a visualization of the contrast between each of the at least one spinal discs and surrounding tissue.

16. The method of claim 15, wherein using fluoroscopy comprises using computed tomography.

17. The method of claim 15, comprising:
injecting additional gadolinium into a spinal disc experiencing gadolinium runoff to indicate an enhanced contrast; and
using fluoroscopy to create an enhanced visualization from the enhanced contrast.

18. A method of using gadolinium to facilitate lumbar discography, comprising:
injecting gadolinium into at least one spinal disc to create a contrast between each of the at least one spinal discs and surrounding tissue;
using computed tomography to identify the contrast and create from the contrast at least one of a two-dimensional visualization and a three-dimensional visualization.

19. The method of claim 18, comprising:
injecting additional gadolinium into a spinal disc experiencing gadolinium runoff to enhance the quality of the contrast;
using computed tomography to identify the enhanced contrast and create from the enhanced contrast at least one of a two-dimensional enhanced visualization and a three-dimensional enhanced visualization.
